(19) United States
(12) Patent Application Publication
(10) Pub. No.: US 2026/0347942 A1
(43) Pub. Date: Nov. 5, 2026

(54) METHOD OF DIAGNOSIS OF CHRONIC KIDNEY DISEASE WITH PHARMACOGENOMICS SUPPORT

(71) Applicant: Machaon Diagnostics, Inc., Berkeley, CA (US)

(72) Inventors: James S. Kain, Berkeley, CA (US); Michael P. Ero, Berkeley, CA (US); James Anderson-Furgeson, Berkeley, CA (US); Jihyun Moon, Berkeley, CA (US)

(73) Assignee: Machaon Diagnostics, Inc., Berkeley, CA (US)

(21) Appl. No.: 19/561,990

(22) Filed: Mar. 10, 2026

Related U.S. Application Data
(63) Continuation of application No. 17/874,258, filed on Jul. 26, 2022, now abandoned.
(60) Provisional application No. 63/225,862, filed on Jul. 26, 2021.

(51) Int. Cl. C12Q 1/6883

(52) U.S. Cl. CPC C12Q 1/6883; C12Q 2600/106; C12Q 2600/118; C12Q 2600/156

(57) ABSTRACT

Methods and systems are provided for integrated genomic analysis of chronic kidney disease (CKD) genes and pharmacogenomic genes in a human subject. A biological sample is obtained from the subject and genomic DNA is extracted from the sample. Genomic regions of one or more CKD genes and pharmacogenomic genes are simultaneously amplified using a multiplex primer pool. Amplified DNA fragments are used to prepare a sequencing library and are sequenced using a next-generation sequencing platform. Sequencing reads are processed by a computer processor to align the reads to a reference genome and identify sequence variants within the amplified genomic regions. Output data identifying detected sequence variants are generated for use in clinical evaluation and management of the subject. In some implementations, the genomic variant data are used to guide pharmacological treatment selection or drug dosage for the subject based on detected pharmacogenomic variants.

### CROSS-REFERENCE TO RELATED APPLICATIONS

The present application is a continuation of U.S. patent application Ser. No. 17/874,258 titled “Method of Diagnosis of Chronic Kidney Disease with Pharmacogenomics Support,” filed on Jul. 26, 2022 which claims the benefit of U.S. Provisional Patent Application No. 63/225,862 titled “Method for Diagnosis of Chronic Kidney Disease with Pharmacogenomics Support,” filed on Jul. 26, 2021, the contents of which are incorporated in this disclosure by reference in their entirety.

### REFERENCE TO AN ELECTRONIC SEQUENCE LISTING

The contents of the electronic sequence listing provided as a file titled Sequence_Listing.xml created Feb. 1, 2023 and is 17,412,000 bytes in size and is herein incorporated by reference in its entirety.

### BACKGROUND

Chronic kidney disease (CKD) is defined as a structural (e.g., cysts, tumors, malformations and atrophies) and/or functional defect that persists longer than three months. Stages of CKD are based on the glomerular filtration rate and albuminuria. The final stage is end-stage renal disease (ESRD). Patients with ESRD require dialysis or kidney transplant.

Globally, CKD is very common with a prevalence of 7-12% of the population for all stages of CKD, depending on the country. Five-year survival rate for ESRD patients receiving dialysis is 40-50%. The five-year survival rate for ESRD patients receiving a kidney transplant from a deceased donor is 86% and improves to 93% when the transplant is from a living donor.

Causes of CKD include diabetes, high blood pressure, glomerulonephritis, and polycystic kidney disease. Ultrasound, biopsy, and genetics may be used to identify the underlying cause of a patient's CKD. Identifying the risk factors and underlying causes is recommended for optimal treatment by guidelines such as Kidney Disease Improving Global Outcomes (KDIGO).

Early treatment generally focuses on managing the risk factors by lowering high blood pressure, blood sugar, and cholesterol. Treatment of CKD patients in general is complex because there are frequently co-morbidities such as diabetes or cardiovascular disease. For those on dialysis, infections become a major concern as well. ESRD patients fortunate enough to have received a kidney transplant require immunosuppressives.

Pharmacogenomics (PGX), also called pharmacogenetics, is a subset of precision medicine where drug choice or drug dose is tailored based on a patient's specific genotype. Drugs are metabolized by the patient's body and in certain cases can be significantly influenced by the patient's genotype. Knowing how a drug may be affected by genotype may allow the physician to use a more appropriate dose that will maximize efficacy while minimizing side effects. This PGX-informed approach stands in contrast to trial-and-error dosing of a patient or a one-size-fits-all dose.

For example, consider the gene CYP3A5 and the immunosuppressive drug tacrolimus. There is a version (or allele) of CYP3A5 that is very common in the normal population that contains a nonfunctional splice variant. This allele is called CYP3A5*3. People who have CYP3A5*3 require less tacrolimus to reach the same internal concentration as people who have another common version called CYP3A5*1.

Despite its great promise, PGX is not yet routinely clinically used. The reasons are complex but likely include test turnaround time, test reimbursement, inertia in adopting new paradigms, complexity of the interpretation of results, and rapidly evolving scientific knowledge.

Current genetic methods for confirming a clinical diagnosis of CKD typically takes longer than two weeks and does not include treatment guidance from PGX. Doctors and patients are always searching for faster testing turnaround times for faster patient diagnosis and better management.

Therefore, there is a need for an improved method to rapidly identify mutations, polymorphisms and other variants in genes underlying CKD coupled with PGX genotyping for optimized treatment in order to identify, diagnose, treat, and assess the prognosis of an individual.

### SUMMARY

The present disclosure relates to genomic testing methods and systems for integrated analysis of genetic variants associated with chronic kidney disease (CKD) and pharmacogenomic drug response. The disclosed methods and systems implement a technical workflow that combines multiplex nucleic acid amplification, high-throughput sequencing, and computational variant identification to produce genomic information useful in clinical management of a subject.

In one aspect, a method is provided for integrated genomic analysis of CKD genes and pharmacogenomic (PGX) genes in a human subject. A biological sample obtained from the subject is processed to extract genomic DNA. A multiplex amplification reaction is prepared using a primer pool containing primer pairs configured to amplify genomic regions of one or more CKD genes and one or more PGX genes. The genomic regions are amplified simultaneously using polymerase chain reaction to generate amplified DNA fragments.

A sequencing library is prepared from the amplified DNA fragments and the fragments are sequenced. Sequencing reads generated from the sequencing platform are processed using a computer processor to align the sequencing reads to a reference genome and identify sequence variants within the amplified genomic regions. Output data identifying the detected sequence variants are generated.

In some implementations, the amplified genomic regions include coding exons of the genes and flanking intronic regions adjacent to the coding exons. The multiplex primer pool may target genes selected from CKD genes listed in Table 1 and PGX genes listed in Table 2. The methods may further include sequencing coverage sufficient to identify single nucleotide variants, insertions, deletions, and structural variants.

In another aspect, a genomic testing system is provided that includes components configured to perform the above workflow. The system includes a nucleic acid extraction component configured to isolate genomic DNA from a biological sample, a multiplex amplification component comprising a primer pool configured to amplify genomic regions of CKD and PGX genes, a sequencing instrument configured to sequence amplified genomic fragments, and a computational analysis component configured to align sequencing reads to a reference genome and identify sequence variants.

In another aspect, a computer-implemented method is provided for guiding treatment of a subject having or suspected of having kidney disease. Genomic variant data generated from sequencing analysis are received and a pharmacogenomic variant associated with metabolism of a therapeutic agent is identified. Genotype information relevant to drug metabolism is determined based on the identified pharmacogenomic variant and the genotype information is provided for use by a healthcare provider in selecting a drug treatment or dosage for the subject. In some implementations, the PGX gene comprises CYP3A5 and the drug treatment comprises tacrolimus.

The disclosed methods and systems provide a technical framework for integrated detection of genetic variants associated with kidney disease and pharmacogenomic drug response using multiplex sequencing workflows and computational analysis.

### DESCRIPTION

According to one embodiment of the present invention, there is provided a method for rapid detection of variants, mutations, structural variants and polymorphisms associated with CKD and PGX. The time for diagnosing or determining the patient's risk of developing CKD and their PGX profile using the method of the present invention can be as early as 48 hours after receipt of the patient's sample. The method of the present invention can also be used to diagnose CKD and determine their PGX profile within 5 days after receipt of the patient's sample. The method involves analysis of 334 CKD genes and 40 PGX genes, referred to herein as the “genes of interest.”

As used in this disclosure, except where the context requires otherwise, the term “comprise” and variations of the term, such as “comprising,” “comprises” and “comprised” are not intended to exclude other additives, components, integers or steps. Thus, throughout this specification, unless the context requires otherwise, the words “comprise,” “comprising” and the like, are to be construed in an inclusive sense as opposed to an exclusive sense, that is to say, in the sense of “including, but not limited to.”

As used in this disclosure, except where the context requires otherwise, the method steps are not intended to be limiting nor are they intended to indicate that each step is essential to the method or that each step must occur in the order disclosed.

As used herein, “sample” refers to any sample that can be from or derived a human patient, e.g., bodily fluids (blood, saliva, urine etc.), biopsy, tissue, and/or waste from the patient. Thus, tissue biopsies, stool, sputum, saliva, blood, lymph, tears, sweat, urine, vaginal secretions, or the like can be used in the method, as can essentially any tissue of interest that contains the appropriate nucleic acids. The sample may be in a form taken directly from the patient. Preferably, the sample may be at least partially purified to remove at least some non-nucleic acid material.

The term “DNA sequence” as used herein refers to chromosomal sequence as well as to cDNA sequence.

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An “amplicon” is an amplified nucleic acid, e.g., a nucleic acid that is produced by amplifying a template nucleic acid by any available amplification method (e.g., PCR, LCR, transcription, or the like).

A “gene” is one or more sequence(s) of nucleotides in a genome that together encode one or more expressed molecules, e.g., an RNA. The gene can include coding sequences that are transcribed into RNA which may then be translated into a polypeptide sequence, and can include associated structural or regulatory sequences that aid in replication or expression of the gene.

A “set” or “pool” of primers or amplicons refers to a collection or group of primers or amplicons, or the data derived therefrom, used for a common purpose, e.g., identifying an individual with a specified genotype (e.g., risk of developing CKD). Frequently, data corresponding to the primers or amplicons, or derived from their use, is stored in an electronic medium.

A “structural variant” refers to a variation of a DNA sequence such as, for example, a deletion, duplication, insertion, translocation or inversion.

A set of oligonucleotide sequences, or primers, have been identified that facilitate the rapid identification of variants, mutations, polymorphisms, and other structural variants associated with CKD and PGX. This set of oligonucleotides detects sequences that are indicative of whether an individual is a carrier for, or has CKD, and their type of PGX profile.

The primers are used to amplify certain genes underlying CKD and PGX. This includes targeting all exons of the genes, plus 5 base pairs of flanking intronic DNA, plus deep intronic and promoter regions and the 5 prime and 3 prime untranslated regions of the genes, and any structural variants known to be associated with CKD and PGX.

The DNA used in the method of the invention can be extracted from any source, such as, for example, whole blood, samples from a buccal swab, or it can be from previously extracted genomic DNA samples. Preferably, the DNA is extracted from whole blood using known techniques. Prior to analysis, extracted DNA can be stored for one month at room temperature or 2-8 °C. For longer storage of up to 2 years, DNA can be stored frozen at <−20 °C to minimize the degradation of nucleic acid.

After extraction, the DNA sample is measured and assessed for purity. 30 ng of genomic DNA, at a minimum concentration of 1.7 ng/μL, is preferable for analysis, but as little as 1 ng is sufficient. The concentration of purified DNA should preferably be adjusted to between 2 ng/μL and 25 ng/μL prior to analysis. Optimal DNA purity is an absorbance ratio (A₂₆₀/A₂₈₀) of 1.80 or greater (typical range is 1.60 to 2.00).

The extracted sample DNA is then amplified. A well-known amplification method, for example, is the polymerase chain reaction (PCR). In PCR, a characteristic piece of the particular nucleotide sequence of interest is amplified with specific primers. If the primer pair finds its target site, a sequence of the genetic material undergoes a million-fold proliferation.

During the PCR process, the DNA generated is used as a template for replication. This sets in motion a chain reaction in which the DNA template is exponentially amplified. PCR can amplify a single or few copies of a piece of DNA by several orders of magnitude, generating millions or more copies of the DNA piece. PCR can be extensively modified to perform a wide array of genetic manipulations, as known by one of skill in the art.

In the method described herein, unique primer pool aliquots are used to amplify the region of interest from the sample. The primer pool aliquots contain forward and reverse primer pairs directed towards one or more of the 334 CKD genes or the 40 PGX genes, as listed in Table 1 and Table 2.

TABLE 1
CKD Genes

| Gene | Cytogenetic | OMIM Gene MIM | |
|---|---|---|---|
| Name | Location | Number | Disease Name |
| ACE | 17q23 | 106180 | Renal tubular dysgenesis |
| ACTN4 | 19q13 | 604638 | Focal Segmental Glomerulosclerosis 1 |
| ADAMTS13 | 9q34.2 | 604134 | Thrombotic thrombocytopenic |
| | | | purpura, familial |
| ADCK4 | | | Nephrotic syndrome, type 9 |
| ADCY10 | | 605205 | Hypercalciuria, absorptive, |
| | | | susceptibility to |
| AGT | 1q42-43 | 106150 | Renal tubular dysgenesis |
| AGTR1 | 3q24 | 106165 | Renal tubular dysgenesis |
| AGXT | 2q37.3 | 604285 | Hyperoxaluria, primary, type 1 |
| AHI1 | 6q23.3 | 608894 | Joubert Syndrome 3 |
| ALMS1 | 2p13.1 | 606844 | Alstrom syndrome |
| ANKS6 | 9q22.33 | 615370 | Nephronophthisis 16 |
| APOA1 | 11q23.3 | 107680 | Amyloidosis, familial visceral, 3 or |
| | | | more types |
| APOE | | | |
| APOL1 | | | |
| APRT | 16q24.3 | 102600 | Adenine phosphoribosyltransferase |
| | | | deficiency |
| AQP2 | 12q12-q13 | 107777 | Diabetes insipidus, nephrogenic |
| ARHGDIA | 17q25.3 | 601925 | Nephrotic syndrome type 8 |
| ARL13B | 3q111 | 608922 | Nephronophtisis, Joubert syndrome 8 |
| ARL6 | 3q11.2 | 608845 | Bardet-biedl syndrome 3 |
| ATP6V0A4 | 7q34 | 605239 | Renal tubular acidosis, distal |
| AVPR2 | Xq28 | 300538 | Nephrogenic syndrome of |
| | | | inappropriate diuresis Diabetes |
| | | | insipidus, nephrogenic |
| B9D2 | 19q13.2 | 611951 | Meckel syndrome type 10 |
| BBS1 | 11q13.2 | 209901 | Bardet-biedl syndrome 1 |
| BBS10 | 12q21.2 | 610148 | Bardet-biedl syndrome 10 |
| BBS12 | 4q27 | 610683 | Bardet-biedl syndrome 12 |
| BBS2 | 16q12.2 | 606151 | Bardet-biedl syndrome 2 |
| BBS4 | 15q24.1 | 600374 | Bardet-Biedl Syndrome 4 |
| BBS5 | 2q31.1 | 603650 | Bardet-Biedl Syndrome 5 |
| BBS7 | 4q27 | 607590 | Bardet-biedl syndrome 7 |
| BBS9 | 7p14.3 | 607968 | Bardet-biedl syndrome 9 |
| BMP4 | 14q22-23 | 112262 | Microphthalmia, syndromic 6 |
| BSND | 1p32.1 | 606412 | Bartter Syndrome, Type 4a, neonatal |
| CA2 | 8q21.2 | 611492 | Osteopetrosis with renal tubular |
| | | | acidosis |
| CASR | 3q13 | 601199 | Neonatal severe primary |
| | | | hyperparathyroidism, Hypocalcemia |
| | | | with 7mmune syndrome |
| CC2D2A | 4p15.32 | 612013 | Joubert syndrome with hepatic defect, |
| | | | Joubert syndrome with oculorenal |
| | | | defect, Meckel Syndrome 6, Jouber |
| | | | syndrome 9, COACH syndrome |
| CD151 | 11p15.5 | 602243 | Nephropathy with pretibial |
| | | | epidermolysis bullosa and deafness |
| CD2AP | | | |
| CEP164 | 11q23.3 | 614848 | Senior-Loken syndrome, |
| | | | Nephronopthisis 15 |
| CEP290 | 12q21.32 | 610142 | Joubert syndrome with oculorenal |
| | | | defect, Bardet-biedl syndrome 14, |
| | | | Joubert Syndrome 5, Meckel syndrome |
| | | | 4, Senior-Loken Syndrome 6 |
| CEP41 | 7q32.2 | 610523 | Joubert's syndrome type 15 |
| CFH | 1q31.3 | 134370 | Complement factor h deficiency |
| CFHR5 | 1q31.3 | 608593 | Nephropathy due to CFHR5 deficiency |
| CFI | 4q25 | 217030 | Complement factor 1 deficiency |
| CHD7 | 8q12.2 | 608892 | CHARGE syndrome |
| CLCN5 | Xp11.23 | 300008 | Dent disease nephrolithiasis type 1 |
| | | | hypophosphatemic Rickets |
| CLCNKB | 1p36.13 | 602023 | Bartter syndrome, type 3 and type 4b |
| CLDN16 | 3q28 | 603959 | Hypomagnesemia 3, renal |
| CLDN19 | 1p34.2 | 610036 | Familial primary hypomagnesemia with |
| | | | hypercalciuria and nephrocalcinosis |
| | | | with severe ocular |
| | | | involvementHypomagnesemia 5, renal, |
| | | | with ocular involvement |
| COL4A3 | 2q36-q37 | 120070 | Alport syndrome |
| COL4A4 | 2q35-q37 | 120131 | Alport syndrome; nephrotic syndrome |
| | | | (FSGS 1) |
| COL4A5 | Xq22.3 | 303630 | Alport syndrome |
| COL4A6 | | 303631 | |
| COQ2 | 4q21.23 | 609825 | Coenzyme q10 deficiency |
| COQ6 | 14q24.3 | 614647 | Coenzyme Q10 deficiency, primary, 6 |
| CREBBP | 16p133 | 600140 | Rubinstein Taybi syndrome type 1 |
| CSPP1 | 8q13.1-q13.2 | 611654 | Meckel syndrome, Joubert syndrome |
| | | | 21 |
| CTNS | 17p13 | 606272 | Cystinosis, nephropathic cystinosis, |
| | | | late-onset juvenile or adolescent |
| | | | nephropathic |
| CUBN | 10p13 | 602997 | Megaloblastic anemia 1-finnish type |
| CUL3 | 2q36.2 | 603136 | Pseudohypoaldosteronism, type 2e |
| CYP11B1 | 8q24.3 | 610613 | Congenital adrenal hyperplasia due to |
| | | | 11-beta-hydroxylase deficiency |
| DGKE | 17q22 | 601440 | Nephrotic syndrome type 7 |
| DHCR7 | 11q13.4 | 602858 | Smith-Lemli-Opitz Syndrome |
| DSTYK | 1q321 | 612666 | Congenital Anomalies of the Kidney |
| | | | and the Urinary Tract |
| EGF | 4q25 | 131530 | Hypomagnesemia 4, renal |
| EYA1 | 8q13.3 | 601653 | Branchio-oto-renal syndrome 1 |
| FAN1 | 15q13.2-q13.3 | 613534 | Interstitial nephritis, karyomegalic |
| FGA | 4q31.3 | 134820 | Amyloidosis, familial visceral, |
| | | | hereditary renal |
| FGF20 | 8p22 | 605558 | Renal hypodysplasiaaplasia 2 |
| FGF23 | 12p13.3 | 605380 | Hypophosphatemic Rickets, autosomal |
| | | | dominant Tumoral Calcinosis, |
| | | | hyperphosphatemic, familial |
| FGFR1 | 8p12 | 136350 | Encephalocraniocutaneous |
| | | | Lipomatosis, Kallmann syndrome 2, |
| | | | kal2 |
| FN1 | 2q34 | 135600 | Glomerulopathy with fibronectin |
| | | | deposits 2 |
| FRAS1 | 4q21.21 | 607830 | Fraser syndrome |
| FREM1 | 9p22.3 | 608944 | Bifid nose with or without anorectal |
| | | | and renal anomalies |
| FXYD2 | 11q23.3 | 601814 | Hypomagnesemia 2, renal |
| GDNF | 5p131-p12 | 600837 | Congenital Anomalies of the Kidney |
| | | | and the Urinary Tract |
| GLA | Xq22.1 | 300644 | Fabry Disease |
| GLIS2 | 16p13.3 | 608539 | Nephronophthisis 7 |
| GPC3 | Xq26.2 | 300037 | Simpson golabi behmel syndrome type |
| | | | 1 |
| GRHPR | 9p13.2 | 604296 | Hyperoxaluria, primary, type 2 |
| GSN | 9q33.2 | 137350 | Amyloidosis, finnish type cerebral |
| | | | amyloid angiopathy, gsn-related |
| HNF1B | 17q12 | 189907 | Renal Cysts and Diabetes Syndrome |
| HSD11B2 | 16q22.1 | 614232 | Apparent mineralocorticoid excess |
| INF2 | 14q32.33 | 610982 | Focal Segmental Glomerulosclerosis 5 |
| | | | Charcot-Marie-Tooth disease E |
| INPP5E | 9q34.3 | 613037 | Joubert syndrome with hepatic |
| | | | defectJoubert Syndrome 1 |
| INVS | 9q31.1 | 243305 | Senior-Loken syndrome, |
| | | | Nephronophthisis 2, infantile |
| IQCB1 | 3q13.33; 3q21.1 | 609237 | Senior-loken syndrome 5 |
| JAG1 | 20p12.2 | 601920 | Alagille syndrome type 1 |
| KANK1 | 9p24.3 | 607704 | Nephroticsyndrome |
| KCNJ10 | 1q23.2 | 602208 | Seizures, sensorineural deafness, |
| | | | ataxia, mental retardation, and |
| | | | electrolyte imbalance (sesame |
| | | | syndrome) |
| KCNJ5 | 11q243 | 600734 | Hyperaldosteronism, familial, type 3 |
| KIF7 | 15q26.1 | 611254 | Acrocallosal syndrome |
| KLHL3 | 5q31.2 | 605775 | Pseudohypoaldosteronism, type 2d |
| LAMB2 | 3p21.31 | 150325 | Nephrotic syndrome, type 5, with or |
| | | | without ocular abnormalities Pierson |
| | | | Syndrome |
| LMX1B | 9q33.3 | 602575 | Nail-patella syndrome |
| LRP4 | 11p11.2 | 604270 | Cenani-lenz syndactyly syndrome |
| LYZ | 12q15 | 153450 | Amyloidosis, familial visceral, renal |
| MAGI2 | 606382 | 191170 | |
| MEFV | 16p13.3 | 608107 | Familial Mediterranean fever |
| MKKS | 20p12.2 | 604896 | Bardet-biedl syndrome 6, McKusick- |
| | | | Kaufman syndrome |
| MKS1 | 17q22 | 609883 | Meckel syndrome 1, Bardet-Biedl |
| | | | syndrome 13 |
| MMACHC | 1p34.1 | 609831 | Methylmalonic aciduria and |
| | | | homocystinuria, cblc type |
| MUC1 | 1q22 | 158340 | Medullary cystic kidney disease 1 |
| MYH9 | 22q13.1 | 160775 | Fechtner syndrome; Epstein Syndrome |
| MYO1E | 15q21-q22 | 601479 | Glomerulosclerosis, focal segmental, 6 |
| NEK8 | 17q11.1 | 609799 | Nephronophthisis 9 Renal-heaptic- |
| | | | pancreatic dysplasia 2 |
| NLRP3 | 1q44 | 606416 | Muckle-wells syndrome Familial cold- |
| | | | induced inflammatory syndrome 1 |
| NOTCH2 | 1p12 | 600275 | Acroosteolysis dominant type, Alagille |
| | | | syndrome 2, Hajdu-Cheney Syndrome |
| NPHP1 | 2q13 | 607100 | Bardet-Biedl syndrome, |
| | | | Nephronophthisis 1 (juvenile), Senior- |
| | | | Loken syndrome, Joubert syndrome 4 |
| NPHP3 | 3q22.1 | 608002 | Senior-Loken syndrome, |
| | | | Nephronophthisis 3 (adolescent), |
| | | | Meckel syndrome 7, Renal-hepatic- |
| | | | pancreatic dysplasia |
| NPHP4 | 1p36 | 607215 | Nephronophthisis 4 Senior-Loken |
| | | | syndrome 4 |
| NPHS1 | 19q13.12 | 602716 | Congenital nephrotic syndrome, |
| | | | Finnish type |
| NPHS2 | 1q25.2 | 604766 | Nephrotic syndrome type 2 |
| NR3C2 | 4q31.1 | 600983 | Pseudohypoaldosteronism Type 1, |
| | | | autosomal dominant Hypertension, |
| | | | Early-Onset, Autosomal Dominant, |
| | | | With Exacerbation in Pregnancy |
| OCRL | Xq25 | 300535 | Dent disease 2 Lowe syndrome |
| OFD1 | Xp22.2 | 300170 | Joubert syndrome type 10, |
| | | | Orofaciodigital syndrome I, Golabi- |
| | | | Behmel syndrome, type 2 |
| PAX2 | 10q24.31 | 167409 | Renal coloboma syndrome, FSGS 7, |
| | | | Papillorenal Syndrome, PAPRS |
| PDSS2 | 6q21 | 610564 | Coenzyme q10 deficiency, primary, 3 |
| PHEX | Xp22.2-p22.1 | 300550 | Hypophosphatemic rickets |
| PKD1 | 16p13.3 | 601313 | Polycystic kidney disease 1 |
| PKD2 | 4q22.1 | 173910 | Polycystic kidney disease 2 |
| PKHD1 | 6p12.3-p12.2 | 606702 | autosomal recessive polycystic kidney |
| | | | disease, Polycystic and hepatic disease |
| PLCE1 | 10q23.3 | 608414 | Nephrotic syndrome type 3 |
| PTPRO | 12p13.3-p13.2; | 600579 | Nephrotic syndrome, type 6 |
| | 12p13-p12 | | |
| REN | 1q32.1 | 179820 | Renal tubular dysgenesis |
| | | | hyperuricemic nephropathy, familial |
| | | | juvenile 2 |
| RET | 10q11.21 | 164761 | Renal agenesis, bilateral |
| ROBO2 | 3p12.3 | 602431 | Vesicoureteral reflux 2 |
| RPGRIP1L | 16q12.2 | 610937 | Meckel syndrome 5, Joubert syndrome |
| | | | 7, COACH syndrome |
| SALL1 | 16q12.1 | 602218 | Townes-Brocks Branchiootorenal-like |
| | | | Syndrome |
| SALL4 | 20q13.2 | 607343 | Acro-renal-ocular syndrome, Duane- |
| | | | Radial Ray Syndrome IVIC Syndrome |
| SCARB2 | 4q21.1 | 602257 | Epilepsy, Progressive Myoclonic, 4 with |
| | | | or without Renal Failure |
| SCNN1A | 12p13.31 | 600228 | Pseudohypoaldosteronism, Type I |
| SCNN1B | 16p12.2 | 600760 | Liddle syndrome |
| SCNN1G | 16p12 | 600761 | Pseudohypoaldosteronism, type 1 |
| | | | Liddle Syndrome |
| SDCCAG8 | 1q43 | 613524 | Senior loken syndrome type 7, Bardet- |
| | | | Biedl Syndrome 16 |
| SEMA3E | 7q21.11 | 608166 | Charge syndrome |
| SIX1 | 14q23.1 | 601205 | Branchio-oto-renal syndrome |
| SIX2 | 2p21 | 604994 | Renal hypodysplasia |
| SIX5 | 19q13.32 | 600963 | Branchio-oto-renal syndrome 2 |
| SLC12A1 | 15q21.1 | 600839 | Bartter syndrome, type 1, antenatal |
| SLC12A3 | 16q13 | 600968 | Gitelman syndrome |
| SLC22A12 | 11q13.1 | 607096 | Hypouricemia, renal, 1, rhuc1 |
| SLC34A1 | 5q35.3 | 182309 | Fanconi renotubular syndrome 2 |
| | | | hypercalcemia, infantile, 2 |
| | | | nephrolithiasis, osteoporosis, |
| | | | hypophosphatemic, 1 |
| SLC34A3 | 9q34.3 | 609826 | Hypophosphatemic rickets with |
| | | | hypercalciuria, hereditary |
| SLC3A1 | 2p21 | 104614 | Cystinuria |
| SLC4A1 | 17q21.31 | 109270 | Renal tubular acidosis, distal |
| SLC4A4 | 4q13.3 | 603345 | Renal tubular acidosis, proximal, with |
| | | | ocular abnormalities and mental |
| | | | retardation |
| SLC5A2 | 16p11.2 | 182381 | Renal glucosuria |
| SLC7A9 | 19q13.1 | 604144 | Cystinuria |
| SLC9A3R1 | 17q25.1 | 604990 | Nephrolithiasis, osteoporosis, |
| | | | hypophosphatemic, 2 |
| SLIT2 | 4p152 | 603746 | Congenital Anomalies of the Kidney |
| | | | and the Urinary Tract |
| SMARCAL1 | 2q35 | 606622 | Schimke 12mmune-osseous |
| | | | dysplasiaSchimke's immunoosseous |
| | | | dystrophy |
| TCTN1 | | | |
| TMEM216 | 11q12.2 | 613277 | Joubert syndrome 2, Meckel syndrome |
| TMEM237 | 2q33.1 | 614423 | Joubert syndrome type 14 |
| | | | Nephronophtisis 11, Meckel Syndrome |
| TMEM67 | 8q22.1 | 609884 | 3, Joubert syndrome 6, COACH |
| | | | syndrome |
| TNFRSF1A | 12p1331 | 191190 | Autosomal dominant periodic fever |
| | | | syndrome |
| TRAP1 | 16p133 | 606219 | Congenital Anomalies of the Kidney |
| | | | and the Urinary Tract |
| TRIM32 | 9q33.1 | 602290 | Bardet-biedl syndrome 11 |
| TRPC6 | 11q22.1 | 603652 | Focal Segmental Glomerulosclerosis 2 |
| TRPM6 | 9q21.13 | 607009 | Hypomagnesemia 1, intestinal |
| TSC1 | 9q34 | 605284 | Tuberous Sclerosis-1 |
| TSC2 | 16p13.3 | 191092 | Tuberous sclerosis-2 |
| TTC21B | 2q24.3 | 612014 | Nephronophthisis 12 |
| TTC8 | 14q31.3 | 608132 | Bardet-Biedl Syndrome 8 |
| TTR | 18q12.1 | 176300 | Amyloidosis, hereditary, transthyretin- |
| | | | related |
| UMOD | 16p12.3 | 191845 | Medullary cystic kidney disease 2 |
| | | | hyperuricemic nephropathy |
| | | | glomerulocystic kidney disease |
| UPK3A | 22q1331 | 611559 | Renal hypodysplasia, urogenital |
| | | | dysplasia |
| VHL | 3p25.3 | 608537 | Von Hippel-Lindau Syndrome |
| VIPAS39 | 14q24.3 | 613401 | Arthrogryposis, renal dysfunction, and |
| | | | cholestasis 2; arcs2 |
| VPS33B | 15q26.1 | 608552 | Arthrogryposis, renal dysfunction, and |
| | | | cholestasis 1; arcs1 |
| WDPCP | 2p15 | 613580 | Bardet-Biedl syndrome |
| WDR19 | 4p14 | 608151 | Senior-Loken syndrome, Short-rib |
| | | | thoracic dysplasia 5, Nephronophthisis |
| | | | type 13, Cranioectodermal dysplasia |
| WDR35 | 2p24.1 | 613602 | Short rib-polydactyly syndrome, |
| | | | Verma-Naumoff type, Short rib- |
| | | | polydactyly syndrome (Jeune's |
| | | | syndrome) type 7, Cranioectodermal |
| | | | dysplasia type 2 |
| WNK1 | 12p13.33 | 605232 | Pseudohypoaldosteronism, type 2c |
| WNK4 | 17q21.2 | 601844 | Pseudohypoaldosteronism, type 2b |
| WT1 | 11p13 | 607102 | Denys-Drash syndrome, Frasier |
| | | | syndrome, Nephrotic syndrome type 4, |
| | | | Wilms tumor type 1 |
| XDH | 2p23.1 | 607633 | Xanthinuria, type i |
| XPNPEP3 | 22q13.2 | 613553 | Nephronophthisis-like nephropathy 1 |
| ZNF423 | 16q12.1 | 604557 | Nephronophthisis-14 Joubert |
| | | | syndrome 16 |
| ABCG2 | | | Susceptibility to Gout |
| ALG1 | | | Congenital disorder of glycosylation, |
| | | | type Ik |
| ALG8 | 11q14.1 | 608103 | Polycystic liver disease 3 with or |
| | | | without kidney cysts |
| ANLN | 7p14.2 | 616027 | Focal Segmental Glomerulosclerosis 8 |
| ANOS1 | Xp22.31 | 300836 | Hypogonadotropic hypogonadism 1 |
| | | | with or without anosmia (Kallmann |
| | | | syndrome 1) |
| AP2S1 | 19q13.32 | 602242 | Hypocalciuric hypercalcemia, familial |
| | | | type 3 |
| APOPT1 | 14q32.33 | 616003 | Mitochondrial Complex 4 deficiency |
| ARHGAP24 | 610586 | | |
| ARMC9 | | | Joubert syndrome 30 |
| ATP6V1B1 | 2p13.3 | 192132 | Renal tubular acidosis with deafness |
| ATP7B | 13q14.3 | 606882 | Wilson Disease |
| ATXN10 | | | Spinocerebellar ataxia 10 |
| B2M | 15q21.1 | 109700 | Amyloidosis, familial visceral |
| B9D1 | 17p11.2 | 614144 | Meckel syndromeMeckel-gruber |
| | | | syndrome type 9 |
| BBIP1 | 10q25.2 | 613605 | Bardet-biedl syndrome 18 |
| BCS1L | 2q35 | 603647 | Mitochondrial complex 3 deficiency, |
| | | | nuclear type 1 |
| BICC1 | | | |
| BMP7 | | 112267 | CAKUT |
| C2CD3 | | | Orofaciodigital syndrome XIV |
| C3 | 19p13.3 | 120700 | Complement component 3 deficiency |
| CACNA1H | | | |
| CCDC28B | | | Bardet-Biedl syndrome 1, modifier of |
| CD46 | | | |
| CDC5L | 6p21 | 602868 | Congenital Anomalies of the Kidney |
| | | | and the Urinary Tract |
| CDKN1C | 11p15.5 | 600856 | Beckwith-wiedemann syndrome |
| | | | IMAGE syndrome |
| CEP104 | 1p36.32 | 616690 | Joubert syndrome 25 |
| CEP83 | 12q22 | 615847 | Nephronophthisis 18 |
| CFAP418 | | | Bardet-Biedl syndrome 21 |
| CFB | | | |
| CFHR1 | | | |
| CFHR3 | | | |
| CHD1L | | | |
| CHRM3 | 1q43 | 118494 | Prune belly syndrome, Eagle-barrett |
| | | | syndrome |
| CHRNA3 | | | Bladder dysfunction, autonomic, with |
| | | | impaired pupillary reflex and |
| | | | secondary CAKUT |
| CLCN2 | | | |
| CLCNKA | 1p36.13 | 602024 | Bartter's Syndrome Type 4b, Neonatal |
| CLDN10 | | | |
| CNNM2 | 10q24.32 | 607803 | Hypomagnesemia type 6 |
| | | | Hypomagnesemia, seizures, and |
| | | | mental retardation |
| COL4A1 | 13q34 | 120130 | Hereditary angiopathy with |
| | | | nephropathy, aneurysms and Msucle |
| | | | cramps (HANAC) |
| COQ8B | 19q13.1 | 615567 | Nephrotic syndrome, type 9 |
| COX10 | 17p12 | 602125 | Mitochondrial Complex 4 deficiency |
| COX14 | 12q13.12 | 614478 | Mitochondrial Complex 4 deficiency |
| COX20 | 1q44 | 614698 | Mitochondrial Complex 4 deficiency |
| COX6B1 | 19p13.12 | 124089 | Mitochondrial Complex 4 deficiency |
| COX8A | 11q13.1 | 123870 | Mitochondrial Complex 4 deficiency |
| CPLANE1 | | | Joubert |
| CPT2 | 1p32 | 600650 | Lethal neonatal carnitine |
| | | | palmitoyltransferase 2 deficiency |
| CRB2 | 9q33.3 | 609720 | FSGS 9 Ventriculomegaly with cystic |
| | | | kidney disease |
| CRKL | | | Congenital Anomalies of the Kidney |
| | | | BMP4 and Urinary Tract (CAKUT) |
| CTU2 | | | |
| CYP24A1 | 20q13.2 | 126065 | Hypercalcemia infantile, 1 |
| CYP27B1 | | | |
| DACH1 | | | |
| DCDC2 | 6p22.3 | 605755 | Nephronophthisis 19 |
| DIS3L2 | 2q37.1 | 614184 | Perlman's syndrome |
| | | | (nephroblastomatosis, gigantism) |
| DLC1 | 604258 | 603089 | |
| DLG1 | 4q22.1 | 600980 | CAKUT |
| DMP1 | | | Hypophosphataemic rickets |
| EHHADH | 3q27.2 | 607037 | Fanconi renotubular syndrome 3 |
| EIF2AK3 | 2p12 | 604032 | Wolcott-Rallison Syndrome |
| EMP2 | 16p13.13 | 602334 | Nephrotic syndrome type 10 |
| ENPP1 | 6q23.2 | 173335 | Hypophosphataemicrickets |
| ETFA | 15q23-q25 | 608053 | Glutaric aciduria 2a |
| ETFB | 19q13.3 | 130410 | Glutaric acidemia 2b |
| ETFDH | 4q32.1 | 231675 | Glutaric acidemia 2c |
| FAH | 15q25.1 | 613871 | tyrosinemia, type 1 |
| FAT1 | 600976 | 604373 | |
| FOXC2 | 16q24.1 | 602402 | Lymphedema-distichiasis syndrome |
| | | | with renal disease and diabetes |
| | | | mellitus |
| FOXP1 | | 605515 | CAKUT |
| FREM2 | 13q13.3 | 608945 | Fraser syndrome |
| FRTS | | | |
| G6PC | 17q21.31 | 613742 | Glycogen storage disease la |
| GALNT3 | 2q24.3 | 601756 | Tumoral calcinosis, |
| | | | hyperphosphatemic, familial, hftc |
| GANAB | | | Polycystic kidney disease 3 |
| GATA3 | 10p14 | 131320 | Hypoparathyroidism, deafness, renal |
| | | | disease syndrome |
| GATM | | | Fanconi renotubular syndrome 1 |
| GLI3 | 6p14.1 | 165240 | Pallister-Hall syndrome |
| GLIS3 | 9p24.2 | 610192 | Diabetes Mellitus, Neonatal, With |
| | | | Congenital Hypothyroidism |
| GNA11 | 19p13.3 | 139313 | Hypocalciuric hypercalcemia, type 2 |
| GREM1 | | | |
| GRIP1 | 12q14.3 | 604597 | Fraser syndrome |
| HNF4A | 20q13.12 | 600281 | Renal cysts and diabetes syndrome |
| | | | Hyperinsulinism due to HNF4A |
| | | | deficiency; Fanconi renotubular |
| | | | syndrome 4, with maturity-onset |
| | | | diabetes of the young |
| HOGA1 | 10q24.2 | 613597 | Primary hyperoxaluria type 3 |
| HOXA13 | 7p15.2 | 142959 | Hand-foot-uterus syndrome |
| HOXD13 | 2q311 | 142989 | Vacterl association with hydrocephalus |
| HPRT1 | Xq26.2-q26.3 | 308000 | Kelley-Seegmiller Syndrome Lesch- |
| | | | Nyhan Syndrome |
| HPSE2 | 10q24.2 | 613469 | Ochoa syndromeUrofacial syndrome 1 |
| IFT122 | 3q21.3-q22.1 | 606045 | Cranioectodermal dysplasia type 1 |
| IFT140 | 16p13.3 | 614620 | Short rib-polydactyly syndrome |
| | | | (Jeune's syndrome) type 9 |
| IFT172 | 2p23.3 | 607386 | Bardet-Biedl syndrome, Short rib- |
| | | | polydactyly syndrome (Jeune's |
| | | | syndrome) type 10 |
| IFT27 | 22q12.3 | 615870 | Bardet-Biedl syndrome 19 |
| IFT43 | 14q24.3 | 614068 | Cranioectodermal dysplasia type 3 |
| IFT74 | | | Bardet-Biedl |
| IKBKAP | 9q31 | 603722 | Familial dysautonomia, Hereditary |
| | | | sensory and autonomic neuropathy |
| | | | type 3, hsan3 |
| ITGA3 | 17q21.33 | 605025 | Interstitial lung disease, nephrotic |
| | | | syndrome, and epidermolysis bullosa, |
| | | | congenital |
| ITGA8 | 10p13 | 604063 | Renal hypodysplasia-aplasia 1 |
| ITGB4 | 17q25.1 | 147557 | Epidermolysis bullosa, junctional, with |
| | | | pyloric atresia |
| KCNJ1 | 11q24.3 | 600359 | Bartter's syndrome, type 2 |
| KCNQ1OT1 | | | Beckwith-Wiedemann syndrome |
| KCTD1 | 18q11.2 | 613420 | Scalp-Ear-Nipple Syndrome |
| KIF12 | | | CAKUT |
| KIF14 | 1q32.1 | 611279 | Meckel syndrome 12 |
| LCAT | 16q22.1 | 606967 | Norum disease |
| LMNA | 1q22 | 150330 | Atypical Werner syndrome |
| | | | Laminopathy type Decaudain- |
| | | | Vigouroux LMNA-related |
| | | | cardiocutaneous progeria syndrome, |
| | | | Restrictive Dermopathy, Lethal |
| LRIG2 | 1p13.1 | 608869 | Ochoa syndrome, Urofacial Syndrome |
| | | | 2 |
| LZTFL1 | 3p21.31 | 606568 | Bardet Biedl syndrome type 17 |
| MAFB | 20q12 | 608968 | Multicentric carpo-tarsal osteolysis |
| | | | with or without nephropathy |
| MAGED2 | Xp11.21 | 300470 | Bartter syndrome, type 5, antenatal, |
| | | | transient |
| MMUT | 6p12.3 | 609058 | Vitamin B12-unresponsive |
| | | | methylmalonic acidemia type mutO |
| MOCOS | | | Xanthinuria |
| MYCN | 2p24.3 | 164840 | Feingold syndrome |
| NEK1 | 4q33 | 604588 | Short rib-polydactyly syndrome |
| | | | (Jeune's syndrome) type 6 |
| NSD1 | 5q35 | 606681 | Sotos syndrome 1; Beckwith- |
| | | | Wiedemann syndrome, BWS |
| NUP107 | 12q15 | 607617 | Nephrotic syndrome, type 11 |
| NUP205 | 7q33 | 614352 | Nephrotic syndrome, type 13 |
| NUP93 | 16q13 | 614351 | Nephrotic Syndrome, Type 12 |
| PBX1 | | 176310 | Congenital anomalies of kidney and |
| | | | urinary tract syndrome with or without |
| | | | hearing loss, abnormal ears, or |
| | | | developmental delay |
| PLCG2 | | | |
| PMM2 | 16p13.2 | 601785 | Congenital disorder of glycosylation, |
| | | | type Ia |
| PTHB1 | | | Bardet-Biedl syndrome 9 |
| SARS2 | 19q13.2 | 612804 | Hyperuricemia, pulmonary |
| | | | hypertension, renal failure and |
| | | | alkalosis |
| SGPL1 | 603729 | | Nephrotic syndrome, type 14 |
| SLC26A1 | | | Nephrolithiasis, calcium oxalate |
| SLC2A2 | 3q26.2 | 138160 | Fanconi-Bickel syndrome |
| SLC2A9 | 4p16.1 | 606142 | Hypouricemia, renal, 2 |
| SLC36A2 | 5q33.1 | 608331 | Hyperglycinuria |
| SLC41A1 | | | |
| SLC5A1 | | | Renal Glucosuria |
| SLC6A19 | 5p15.33 | 608893 | Hartnup Disorder Hyperglycinuria |
| SLC6A20 | 3p21.31 | 605616 | Hyperglycinuria |
| SLC7A7 | 14q11.2 | 603593 | Lysinuric protein intolerance |
| SMC1A | Xp11.22 | 300040 | Cornelia de Lange syndrome |
| SOX17 | 8q11.23 | 610928 | Vesicoureteral Reflux 3 |
| SOX18 | 20q13.33 | 601618 | Hypotrichosis-lymphedema- |
| | | | telangiectasia-renal defect syndrome |
| SRGAP1 | | 606523 | |
| TBX18 | 6q14.3 | 604613 | Congenital hydronephrosisCongenital |
| | | | anomalies of Kidney and Urinary Tract |
| | | | 2 |
| TCTN2 | 12q24.31 | 613846 | Meckel-gruber syndrome type 8, |
| | | | Joubert syndrome 24 |
| TCTN3 | 10q24.1 | 613847 | Joubert syndrome 18, Orofaciodigital |
| | | | syndrome 4 |
| TFAP2A | 6p24.3 | 107580 | Branchiooculofacial Syndrome |
| THBD | | | |
| TMEM107 | | | |
| TMEM138 | qq112.2 | 614459 | Joubert syndrome 16 |
| TNXB | 6p21.33-p21.32 | 600985 | Vesicoureteral Reflux 8 Ehlers-Danlos |
| | | | syndrome due to tenascin-X deficiency |
| TP63 | 3q28 | 603273 | EEC syndrome, Ectrodactyly, |
| | | | ectodermal dysplasia, and cleft |
| | | | lip, palate syndrome 3 |
| VDR | 601769 | | |
| WDR73 | 15q25.2 | 616144 | Galloway-Mowat syndrome |
| WFS1 | 4p16.1 | 606201 | Wolfram syndrome type 1 |
| WNT4 | 1p36.12 | 603490 | SERKAL syndrome Mullerian aplasia |
| | | | and hyperandrogenism |
| XPO5 | 607845 | | |
| ACTG1 | | | Congenital anomalies of kidney and |
| | | | urinary tract |
| ANKFY1 | 607927 | | |
| ATP6B1 | | | Distal renal tubular acidosis 2 with |
| | | | progressive sensorineural hearing loss |
| FAM20A | 17q24.2 | 611062 | Amelogenesis imperfecta- |
| | | | nephrocalcinosis |
| | | | syndromeAmelogenesis imperfecta |
| | | | type IG (enamel-renal syndrome) |
| FGF10 | 5p13-p12 | 602115 | Lacrimoauriculodentodigital syndrome |
| ITSN2 | 604464 | 164860 | |

TABLE 2
PGX Genes
Gene Name

ABCG2
ADRA2A
ANKK1
APOE
COMT
CYP1A2
CYP2C19
CYP2C9
CYP2D6
CYP3A4
CYP3A5
DPYD
F2
F5
GRIK4
HTR2A
HLA-B
MTHFR
OPRM1
SLCO1B1
TPMT
VKORC1
ABCB1
CYP2B6
CYP2C8
DBH
DRD1
DRD4
GABRA6
GABRP
HLA-A
HCP5
HTR2C
ITGB3
KIF6
OPRD1
OPRK1
UGT1A1
UGT2B15
UGT2B7

The primers used to amplify the sequences are listed in the sequence listing.

Following standard techniques, the amplified sequences, or amplicons, are purified and a library is made with the purified amplicons. The library contains multiple sequences from different areas of the 374 total genes of interest. The library made from the amplicons is then amplified and sequenced.

The DNA sequence of the library may be determined by any suitable method. For example, the DNA sequence may be determined by Sanger sequencing (chain termination), pH sequencing, pyrosequencing, sequencing-by-hybridization, sequencing-by-ligation, etc. Exemplary sequencing systems include pyrosequencing (454 Life Sciences), Illumina (Solexa) sequencing, sequencing by ligation (SOLID, Applied Biosystems), long read sequencing (PacBio or Oxford Nanopore Technologies) and Ion Torrent Systems' pH sequencing system.

After sequencing, the DNA sequence of the library is mapped with one or more reference sequences to identify sequence variants. For example, the base reads are mapped against a reference sequence, which in various embodiments is presumed to be a “normal” non-disease sequence. The human reference genome sequence (e.g., hg19 or hg38) is generally used as the reference sequence. A number of computerized mapping applications are known, and include GSMAPPER, ELAND, MOSAIK, and MAQ. Various other alignment tools are known, and could also be implemented to map the DNA sequence of the amplicon library.

Based on the sequence alignments and mapping results, plus available information including, for example, information from human mutational databases and other reference databases, sequence variants in the amplified amplicons are identified. Copy number (i.e., deletions or duplications) may be inferred by analyzing the relative amplicon read depths; deletions will have a statistically significant decrease in relative amplicon read depth spanning the deleted region while duplications will have a statistically significant increase in relative amplicon read depth spanning the duplicated region. Furthermore, any sequence variations, including variants associated with CKD or PGX, disease-associated polymorphisms, benign polymorphisms and other known variants of undetermined significance can be determined to be homozygous, heterozygous, or hemizygous. Any variations in the gene analyzed as compared to the normal control gene can be classified as pathogenic, likely pathogenic, uncertain, likely benign or benign, as recommended by the American College of Medical Genetics (ACMG).

### EXAMPLES

### Example 1

DNA was extracted from the whole blood of Patient 1 using known procedures. The extracted sample DNA was then PCR amplified. The complete set of two unique primer pool aliquots were used with the extracted sample DNA. In the present example, the 374 target genes were amplified and analyzed.

Following amplification of the DNA, the amplicons were purified. A library was then made with the purified amplicons. The library was amplified and sequenced. After sequencing, the sequence of the amplicons from the sample were compared to a control reference sequence comprising the human genome (hg38) sequence.

A heterozygous mutation in the COL4A4 gene was identified in Patient 1. The patient has a very rare heterozygous nonsense mutation (c.2906C>G, p.Ser969Ter) in exon 32 of the COL4A4 gene. This mutation has a minor allele frequency of 0.00006 in the ExAC database. Truncation mutations such as this one are typically damaging to the protein. It is listed as pathogenic 11 times in the LOVD Gene Variant database. It was found in Alport patients with renal failure between the ages of 20 and 30; four were homozygous and one was heterozygous. This mutation is enriched, particularly in patients of British origin.

### Example 2

DNA was extracted from the whole blood of Patient 2 using known procedures. The extracted sample DNA was then PCR amplified. In the present example, the 374 target genes were amplified and analyzed.

Following amplification, the amplicons were purified. A library was then made with the purified amplicons. The library was amplified and sequenced. After sequencing, the sequence of the amplicons from the sample were compared to a normal, control reference sequence comprising the hg38 sequence.

A common polymorphism, c.12627T>G, p.Pro4210Pro, was detected in the PKD1 gene from Patient 2. This polymorphism is present in about 20% of the general population. The polymorphism was detected in 48% of the total reads, indicating this donor is heterozygous for this variant. This polymorphism does not correlate with CKD.

Although the present invention has been discussed in considerable detail with reference to certain preferred embodiments, other embodiments are possible. Therefore, the scope of the appended claim should not be limited to the description of preferred embodiments contained in this disclosure. All references cited herein are incorporated by reference in their entirety.

What is claimed is:

1. A method for integrated genomic analysis of chronic kidney disease (CKD) genes and pharmacogenomic (PGX) genes in a human subject, comprising:
(a) receiving a biological sample obtained from the human subject;
(b) extracting genomic DNA from the biological sample;
(c) preparing a multiplex amplification reaction comprising a primer pool containing primer pairs configured to amplify genomic regions of:
(i) one or more genes selected from the CKD genes listed in Table 1 of the specification; and
(ii) one or more genes selected from the PGX genes listed in Table 2 of the specification;
(d) simultaneously amplifying the genomic regions using polymerase chain reaction to generate amplified DNA fragments;
(e) preparing a sequencing library from the amplified DNA fragments;
(f) sequencing the amplified DNA fragments;
(g) processing sequencing reads using a computer processor to:
(i) align the sequencing reads to a reference genome; and
(ii) identify sequence variants within the amplified genomic regions; and
(h) generating output data identifying detected sequence variants within the amplified genes.

2. The method of claim 1, wherein the biological sample comprises whole blood.

3. The method of claim 1, wherein the extracted genomic DNA comprises between 1 ng and 30 ng of DNA.

4. The method of claim 1, wherein the amplified genomic regions comprise coding exons of the genes and flanking intronic regions adjacent to the coding exons.

5. The method of claim 4, wherein the flanking intronic regions comprise at least 5 base pairs adjacent to the coding exons.

6. The method of claim 1, wherein the genes selected from Table 1 comprise at least 50 genes associated with kidney disease.

7. The method of claim 1, wherein the genes selected from Table 2 comprise one or more PGX genes associated with drug metabolism.

8. The method of claim 7, wherein the PGX gene comprises CYP3A5.

9. The method of claim 1, wherein sequencing of the amplified DNA fragments produces sequence coverage of at least 100× across the amplified genomic regions.

10. A genomic testing system configured to detect genetic variants associated with chronic kidney disease and pharmacogenomic drug response in a human subject, the system comprising:
(a) a nucleic acid extraction component configured to isolate genomic DNA from a biological sample;
(b) a multiplex amplification component comprising a primer pool configured to amplify genomic regions of:
(i) genes selected from the CKD genes listed in Table 1 of the specification; and
(ii) genes selected from the PGX genes listed in Table 2 of the specification;
(c) a sequencing instrument configured to sequence amplified genomic fragments generated from the multiplex amplification component; and
(d) a computational analysis component configured to:
(i) align sequencing reads to a reference genome; and
(i) identify sequence variants within the amplified genomic regions.

11. The system of claim 10, wherein the multiplex amplification component comprises primers targeting coding exons and adjacent intronic regions of the genes.

12. The system of claim 10, wherein the sequencing instrument comprises a next-generation sequencing platform.

13. The system of claim 10, wherein the computational analysis component is configured to detect single nucleotide variants, insertions, deletions, or structural variants.

14. The system of claim 10, wherein the multiplex amplification component is configured to amplify at least 300 CKD genes and at least 30 PGX genes.

15. The system of claim 10, wherein the computational analysis component is configured to generate output data identifying detected sequence variants within the amplified genes.

16. A computer-implemented method for guiding treatment of a subject having or suspected of having kidney disease, comprising:
(a) receiving genomic variant data generated according to the method of claim 1;
(b) identifying a pharmacogenomic variant within a pharmacogenomic gene associated with metabolism of a therapeutic agent;
(c) determining genotype information relevant to drug metabolism based on the identified pharmacogenomic variant; and
(d) providing the genotype information for use by a healthcare provider in selecting a drug treatment or dosage for the subject.

17. The method of claim 16, wherein the PGX gene comprises CYP3A5.

18. The method of claim 17, wherein the drug treatment comprises tacrolimus.

19. The method of claim 16, wherein the genomic variant data is generated within 48 hours of receiving the biological sample.

20. The method of claim 16, wherein the genotype information is used to adjust a dosage of an immunosuppressive drug administered to the subject.
